(19) United States
(12) Patent Application Publication
(10) Pub. No.: US 2009/0012411 A1
(43) Pub. Date: Jan. 8, 2009

(54) Method and apparatus for obtaining electronic oscillotory pressure signals from an inflatable blood pressure cuff

(76) Inventors: Andrew Lowe, Auckland (NZ); Daniel Norberto Roldan, Auckland (NZ); Nigel E. Sharrock, New York, NY (US)

Correspondence Address: MILDE & HOFFBERG, LLP, 10 BANK STREET, SUITE 460, WHITE PLAINS, NY, 10606, US

(21) Appl. No.: 12/157,854

(22) Filed: Jun. 13, 2008

Related U.S. Application Data
(60) Provisional application No. 60/936,495, filed on Jun. 20, 2007.

(51) Int. Cl. A61B 5/0225

(52) U.S. Cl. 600/495

(57) ABSTRACT

A method and apparatus are disclosed for the non-invasive measurement, processing and utility of blood pressure cuff oscillation signals from a blood pressure cuff applied to a body part when the blood pressure cuff pressure is at supra-systolic pressures. Oscillometric cardiac pulse waveforms associated with the peripheral artery are monitored during a plurality of cardiac ejection cycles while at supra-systolic pressure, and are measured pneumatically and transduced by an oscillometric pressure sensor. The analog signal is amplified, processed and digitized to provide a recordable waveform that can be analyzed to obtain information relating to the patient's cardiovascular status.

### CROSS-REFERENCE TO RELATED APPLICATIONS

This application claims priority from U.S. provisional application No. 60/936,495, filed Jun. 20, 2007.

This application also contains subject matter that is disclosed in U.S. Pat. No. 6,994,675, issued Feb. 7, 2006 and entitled “Non-Invasive Measurement of Supra-Systolic Signals,” U.S. patent application Ser. No. 11/358,283 filed Feb. 21, 2006 and entitled “System And Method For Non-Invasive Cardiovascular Assessment From Supra-Systolic Signals Obtained From A Wideband External Pulse Transducer In A Blood Pressure Cuff,” Publication No. US 2006/0224070 A1, (“the '070 publication”), and U.S. patent application Ser. No. 11/495,279, filed Jul. 28, 2006. The subject matter of this patent and these two applications are incorporated herein by reference.

### BACKGROUND OF THE INVENTION

It has been known that the peripheral arterial pressure waveform carries information useful for evaluating a person's health and cardiovascular status and performance. The signals recorded from a blood pressure cuff are termed “supra-systolic” signals if the cuff pressure is above the subject's systolic blood pressure. In addition, signals can be recorded when the cuff pressure is below systolic pressure. In all cases, the signals result from pressure energy transmissions and are dependent upon the subject's physiology.

When the heart pumps, a pressure gradient is generated within the cardiovascular system. This results in pulse pressure waves traveling peripherally from the heart through the arteries. Like any wave, they reflect back off a surface or other change in impedance. Arterial pulse waves reflect back from both the peripheral circulation and from the distal aorta when it becomes less compliant. See Murgo, J. P. et al., “Aortic Input Impedence In Normal Man; Relationship to Pressure Waveforms,” Circulation 62, No. 1 (1980), and Latham, R. D. et al., “Regional Wave Travel and Reflections Along the Human Aorta,” Circulation 72, No. 6 (1985). These reflection waves are identifiable in arterial pressure tracings, but the exact timing and magnitude of the waves are difficult to discern. Nevertheless, they have been the basis of several commercial systems to assess reflectance waves. These systems measure arterial contours using applanation tonometry from the radial artery.

If a low frequency sensor is placed over the brachial artery beneath a blood pressure cuff and the cuff is inflated above systole, supra-systolic signals can be recorded. See Blank, S. G. et al., “Wideband External Pulse Recording During Cuff Deflation,” Circulation 77, No. 6 (1988); Hirai, T. et al., “Stiffness of Systemic Arteries in Patients with Myocardial Infarction,” Circulation 80: 78-86 (1989); and Denby, L. et al., “Analysis of the Wideband External Pulse,” Statistics in Medicine, John Wiley & Sons (1994). An idealized supra-systolic signal for one heart beat is shown in FIG. 1 of the '070 published application. These signals contain frequency components of less than 20 Hertz, which are non-audible. Supra-systolic low frequency signals provide clear definition of three distinct waves: an incident wave corresponding to the pulse wave and two subsequent waves. Blank (Blank, et al., Association of the Auscultatory Gap, Ann. Intern. Med. 124 (10): 877-883 (May 1996)) proposed that the second wave emanated from the periphery and the relative amplitude of this wave to the incident wave (K1R) was a measure of peripheral vascular resistance (PVR). He proposed a constant such that PVR could be measured from the ratio of the incident to the first reflectance wave. See also, U.S. Pat. No. 5,913,826, which is incorporated herein by reference in its entirety.

The second supra-systolic wave is, in fact, a reflectance wave from the distal abdominal aorta—most likely originating from the bifurcation of the aorta and not from the peripheral circulation as proposed by Blank. This has been verified in human experiments (Murgo, Westerhof et al. 1980; Latham, Westerhof et al. 1985) and in studies using pulse wave velocity (PWV) measurements. The relative amplitude of the first reflectance wave is now believed to be a measure of the stiffness, compliance, or elasticity of the abdominal aorta rather than peripheral resistance.

The third wave occurs at the beginning of diastole and is believed to be a reflection wave from the peripheral circulation. As such, it is a measure of peripheral vasoconstriction with superimposed secondary reflections. Supra-systolic signals can be utilized to measure compliance by relating the amplitude of the first wave (incident or SS1) to the amplitude of the second (aortic reflection or SS2) wave. The degree of vasoconstriction can be assessed by measuring the amplitude of the diastolic or third wave (SS3 wave) and relating it to the SS1 wave. Amplitudes, areas under the curves, or other values calculated from the waves can be utilized. Data has been analyzed by measuring amplitudes, ratios of amplitudes and time delays between waves.

Many measurement techniques and methods of interpretation have been proposed in the literature. The most well known is the use of the oscillatory part of a cuff pressure signal for the estimation of peripheral blood pressure.

More recently, there has been interest in attempts to use other sources of pulse waves for the evaluation of the cardiovascular status of a subject. The most direct measurement technique involves the applanation tonometry of an artery, for example, the radial. Plethysmographic sensors have also been employed.

In the case of applanation tonometry, the transduction of a good-quality signal requires a high degree of training and skill. Attempts have been made to automate this procedure, however, the cost of doing so is high, and performance still leaves much to be desired in practice.

Plethysmographic sensing (optical, electrical or mechanical) has also been employed. However, these methods do not measure pressure changes directly, and so require significant, error-prone mathematical transformation to estimate the arterial pressure wave. In addition, the measurement site is often at the finger, whereas pressures of interest are generally more proximal, e.g., in the brachial or radial arteries. (Medically speaking, the brachial pressures have the most clinical literature associated with them.)

The use of a wide-band external pulse sensor to record mechanical vibrations has also been proposed. In U.S. Pat. No. 5,913,826, Blank describes the use of such a system to first estimate the arterial pressure waveform, and secondly calculate indices related to cardiovascular status. Sharrock proposes a variation in U.S. Pat. No. 6,994,675 wherein the information is gained from the wide-band external pulse sensor with an occluding cuff inflated to supra-systolic pressures.

As an alternative to the aforementioned methods, it has been proposed that the readily available, oscillometric signal from a pressure cuff be used to infer information about a person's health.

There are two main problems with using a cuff pulse waveform as a basis for deriving information about a person's health. There is generally found instability and irreproducibility in the measurement of a cuff-pulse wave. In particular, the cuff pulse wave changes shape with cuff pressure. Also, the measured cuff pulse wave does not correspond to the intra-arterial pulse wave shape.

The present invention provides a solution to the above problems by providing a waveform that is both stable and repeatable. It is thus useful as the basis for calculating further indices related to the cardiovascular status of the subject. With regard to the second problem, the measurement process is such that there is no need to scale or correct the acquired waveform in order to extract a signal that resembles the intra-arterial pulse wave shape.

In addition to the above features, the invention may be implemented easily without adding significant cost or complexity above the current oscillometric blood pressure methods, while retaining all the benefits of oscillometric measurement methods over applanation tonometry, plethysmography and wide-band external pulse sensors.

### OBJECTS OF THE INVENTION

It is therefore an object of the invention to provide non-invasive cardiovascular assessment.

It is also an object of the invention to provide non-invasive assessment of aortic compliance by analysis of supra-systolic signals.

It is a further object of the invention to provide a system for assessing cardiovascular status non-invasively, said system comprising an external peripheral pressure cuff, an oscillometric pressure sensor, an analog signal processing circuit including an amplifier, an A/D converter and a digital signal processor.

It is a yet further object of the invention to provide a method for determining a compliance value comprising the steps of measuring a pressure waveform of a peripheral artery with blood flow occluded, measuring the difference in amplitude between a first supra-systolic peak and first supra-systolic trough, measuring the difference in amplitude between a second supra-systolic peak and a second supra-systolic trough, and determining the ratio of the two differences.

It is still another object according to the present invention to provide a non-invasive cardiac monitoring system comprising a blood pressure cuff, a pressure control system for said cuff, a cuff oscillation sensor for measuring pulse waves from the cuff at supra-systolic pressure, and a system for analyzing an output from the cuff oscillation sensor to produce data indicative of cardiac status.

These and other objects of the invention will become more apparent in the description below.

### SUMMARY OF THE INVENTION

The invention concerns the measurement, processing and utility of cuff oscillation signals recorded from a pressure cuff applied to a body part when the cuff pressure is at supra-systolic pressure.

The present invention therefore provides a system for measuring peripheral arterial signals, e.g. of the brachial artery, using an external peripheral pressure cuff, an oscillometric pressure sensor which transduces the waveform pulse signal to a linear analog signal, an analog signal processing circuit which differentially amplifies the analog signal and further amplifies a comparative signal, an A/D converter and a digital signal processor, to produce a waveform with respect to an inferred original aortic waveform.

The systolic pressure of the patient is measured. A cuff is inflated to a determined supra-systolic pressure, such as 15-150 mm Hg above a systolic pressure, preferably about 30 mm Hg above the systolic pressure, measuring with a oscillometric pressure sensor/transducer having sufficient bandwidth to capture detailed waveform information, for example from 0.1 to 1000 Hz, and analyzing the waveform to infer an aortic pressure waveform.

This inferred waveform may then be used for a number of purposes, including analyzing cardiac function, analyzing the central and/or peripheral arterial system, or for analyzing the cardiovascular system as a whole.

Thus, the present invention provides means for extracting useful parameters of central and peripheral cardiovascular system performance, without requiring a direct measurement of waveforms from the heart or aorta.

A reliable system may therefore be provided to acquire supra-systolic signals from patients, a method to analyze the signals, and clinical applications for the signals. The system consists of an oscillometric pressure sensor pneumatically connected to a blood pressure cuff or similar device, placed around a patient's arm. The signals are conditioned and differentially amplified, passed through an analog to digital converter and transferred to a computer or processor for analysis. Analyzed signals will be stored, presented on a screen numerically or graphically. Data can be stored or transmitted to databases or other health care facilities.

A variety of oscillometric pressure sensors can be used. The pressure sensors must be able to sense dynamic pressure signals as low 0 to about 50 kPA (0 to 7.25 psi), have a full scale span of about 40 mV, and be sturdy enough to withstand repeated use under external pressures of about 300 mm Hg. For example, a suitable commercially available piezoresistive pressure sensor is available from Freescale Semiconductor, Inc., as model MPXV2053G, Case 1369-01.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is a schematic block diagram of apparatus in accordance with an embodiment of the present invention;

FIG. 2 is a schematic block diagram of apparatus in accordance with a preferred embodiment of the present invention, showing an oscillometric NIBP measurement module;

FIG. 3 is a further detailed schematic block diagram of apparatus in accordance with the preferred embodiment of the invention shown in FIG. 2;

FIG. 4 is a schematic block diagram of apparatus in accordance with the preferred embodiment of the invention shown in FIG. 3, providing further detail of the great board;

FIG. 5 is a schematic block diagram of the function of an analog signal processing circuit of an embodiment of the invention;

FIG. 6A is an oscilloscope recording of an overall supra-systolic signal;

FIG. 6B is an oscilloscope recording of a supra-systolic signal showing the supra-systolic region;

FIG. 6C is an oscilloscope recording of a supra-systolic signal showing the supra-systolic beat;

FIG. 7 is pressure profile showing the reference pressure provided by the analog signal processing circuit and the actual cuff pressure;

FIG. 8 is a schematic block diagram of apparatus in accordance with a preferred embodiment of the invention;

FIG. 9 shows a pressure trace of a sequence of consecutive supra-systolic beats recorded after amplification from the oscillometric pressure sensor, and where the beats are superimposed upon each other; and

FIG. 10 shows a further pressure trace of FIG. 9, showing a waveform average.

### DETAILED DESCRIPTION OF THE PREFERRED EMBODIMENTS

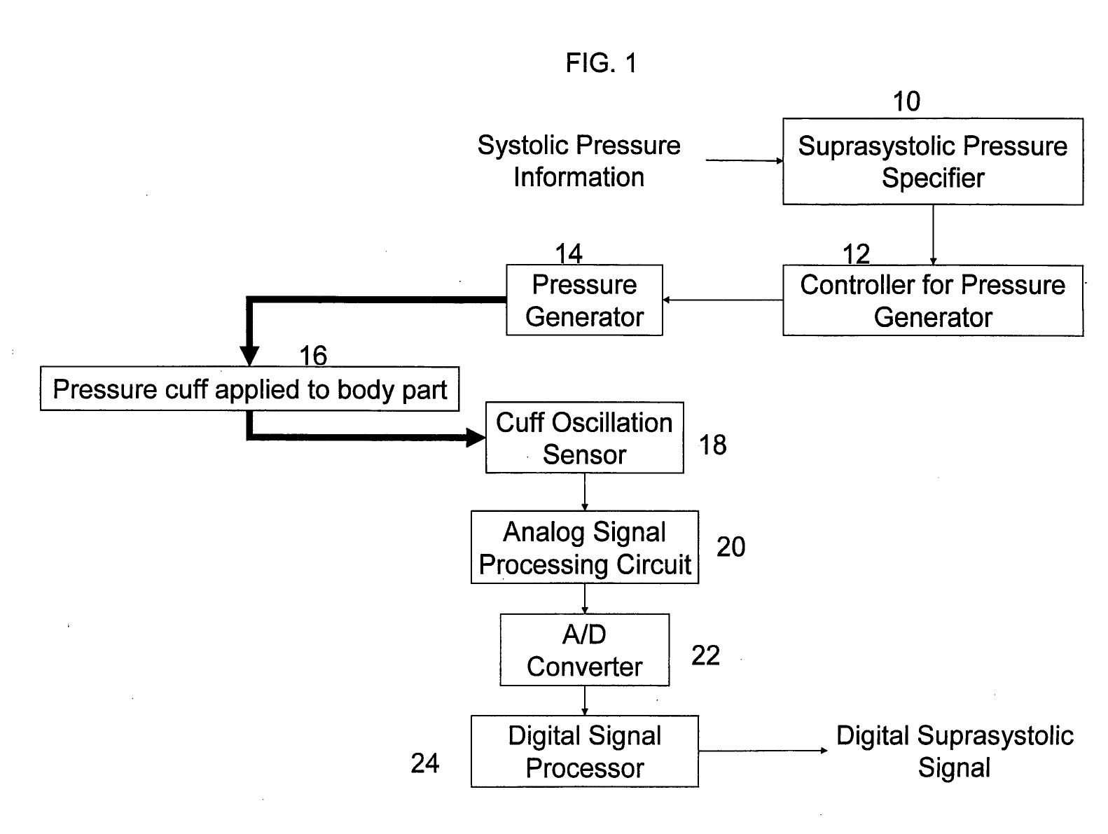

The preferred embodiments of the present invention will now be described with reference to FIGS. 1-10 of the drawings. Identical elements in the various figures are designated with the same reference numerals.

This invention concerns the measurement, processing and utility of blood pressure cuff oscillation signals recorded from a blood-pressure cuff applied to a body part, when the cuff pressure is above the systolic pressure, i.e., at supra-systolic pressures. The amplitude of such signals is typically much less than the total amplitude of cuff oscillation signals. However, these signals carry information directly related to the intra-arterial pressure waveform. Further, at supra-systolic pressures the artery is always in an occluded state, rendering the system being measured relatively constant. The cuff pulse pressure wave is therefore consistent and reproducible, and comparable between subjects.

With reference to the drawings and in particular FIG. 1 initially, a general diagram of an apparatus for pneumatically measuring cuff pulse waves at a supra-systolic pressure, and manipulating the resulting analog signal into a digital supra-systolic signal is shown. Initially, the supra-systolic pressure at which the cuff pulse waves are to be measured must be determined. This is accomplished by first obtaining systolic pressure information, which can be done in a manner described in the '070 publication (incorporated herein by reference). Once the systolic pressure information is obtained, the supra-systolic pressure at which the cuff pulse waves are to be recorded is designated by supra-systolic pressure specifier 10. The supra-systolic pressure information from supra-systolic pressure specifier 10 is fed into a controller 12 for a pressure generator 14 that pneumatically generates the supra-systolic pressure at blood pressure cuff 16. Cuff 16 may be affixed to any appropriated human body part, such as an arm or leg, but typically the arm is preferred. Accordingly, pressure generator 14 provides air pressure through a pneumatic tube (not shown) which inflates cuff 16 to the desired supra-systolic pressure as designated by controller 12.

Once cuff 16 is inflated to the desired supra-systolic pressure, pneumatic signals relating to cuff pulse waves are measured through the pneumatic tube by cuff oscillation sensor 18. Cuff oscillation sensor 18 senses the pneumatic pressure waves generated from cuff 16. Commercially available pressure sensors can be used for this purpose. For example, silicon pressure sensors from Freescale Semiconductor, Inc., such as silicon pressure sensors of series MPX2053/MPXV2053G are preferred. These silicon piezoresistive pressure sensors provide a highly accurate and linear output directly related to the pressure applied. The preferred procedure for measuring the cuff pulse waves is further described below.

Thus, cuff oscillation sensor 18 transduces the pneumatic cuff pulse wave signals to an analog electrical signal corresponding to the pneumatic cuff pulse waves, which are then sent to analog signal processing circuit 20, which processes and amplifies the signal, as further described below. The processed analog signal is then converted to a digital signal by A/D converter 22, which digital signal is, in turn, processed by digital signal processor 24. The result is a digital supra-systolic pressure signal that can be recorded on a chart in a manner known to those of skill in the art, and utilized to determine the cardiovascular status of a patient as described, inter alia, in the '070 publication.

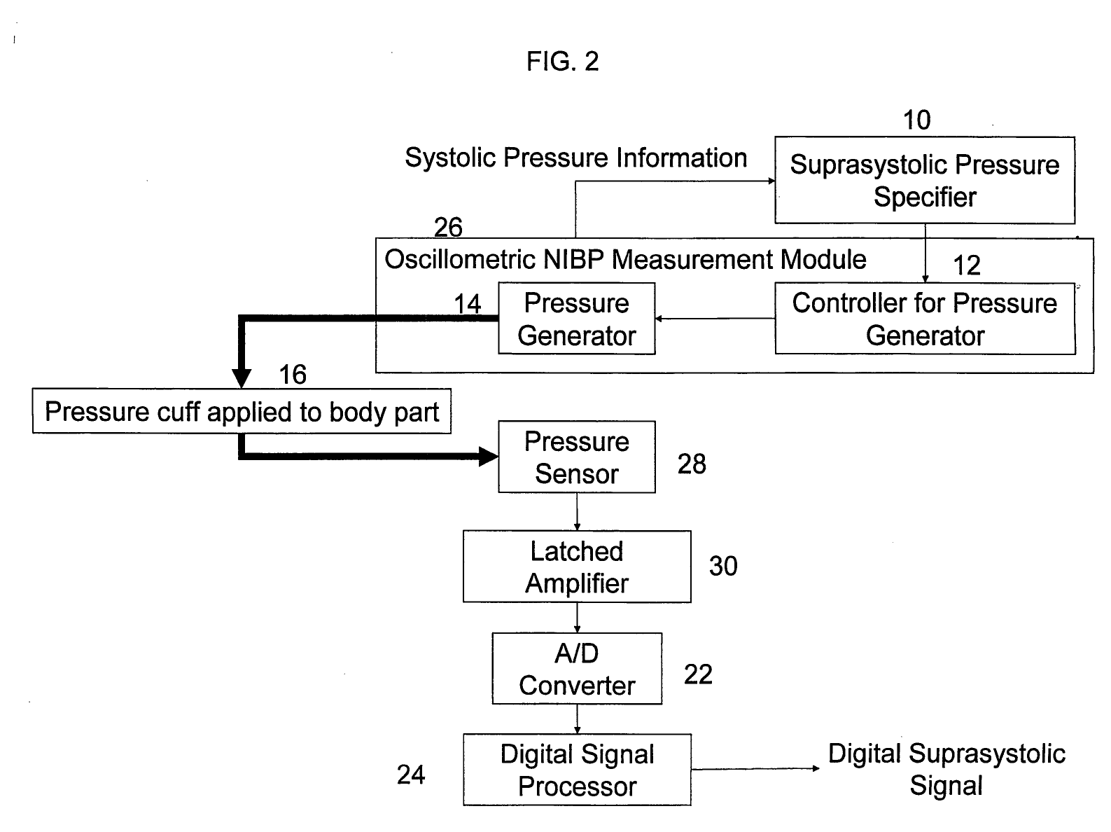

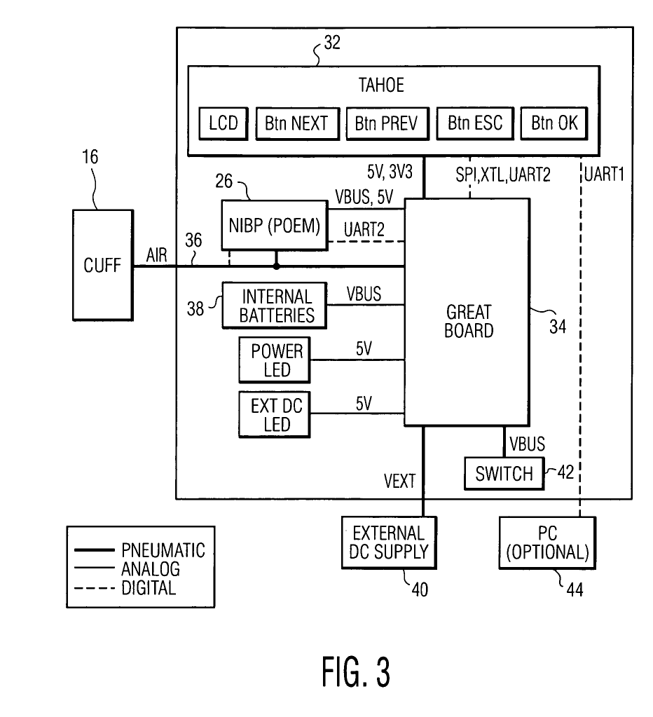

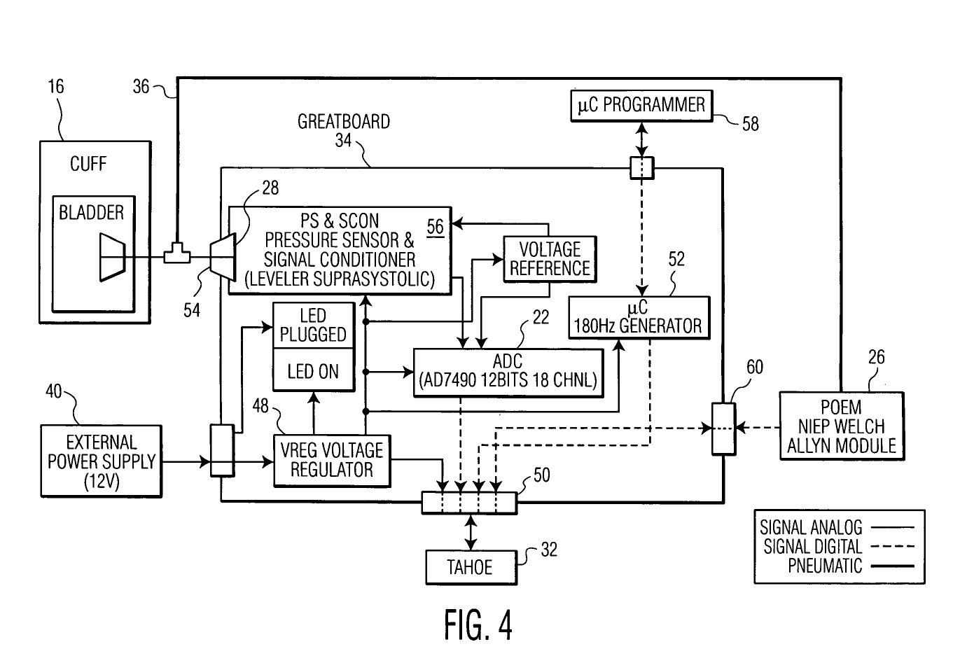

FIG. 2 illustrates a further general diagram of a preferred embodiment of the invention. This embodiment includes an aspect for the oscillometric, non-invasive blood pressure (“NIBP”) determination of the systolic pressure of the subject. The systolic pressure measurement is taken immediately prior to the supra-systolic pressure measurement. As described above, pressure generator 14, operated by controller 12, both contained within oscillometric NIBP measurement module 26, maintain the cuff pressure at any desired pressure. In this embodiment, oscillometric NIBP measurement module 26 operates to control the cuff pressure in a manner for determining the systolic pressure of a patient. Referring to FIGS. 3 and 4, the cuff pressure, in this instance systolic pressure, is measured by the NIBP measurement module 26. The systolic pressure measurement signal is then input into supra-systolic pressure specifier 10 (TAHOE 32, as shown in FIGS. 3 and 4). Alternatively, the systolic pressure of the patient can be determined by any suitable method and the systolic pressure information is input into supra-systolic pressure specifier 32. Supra-systolic pressure specifier 32 then determines the supra-systolic pressure for measuring the cuff pulse waves. It is preferred that the supra-systolic pressure used is 15-30 mm Hg above the determined systolic pressure. It is more preferred that the supra-systolic pressure used is 25-30 mm Hg above the determined systolic pressure. However, any suitable pressure increment above systolic pressure may be determined based on other information such as cuff size, limb size, body mass index, etc.

Once the supra-systolic pressure is determined, oscillometric NIBP measurement module 26 operates to achieve the supra-systolic pressure in cuff 16 for at least one heartbeat. Accordingly, NIBP measurement module 26 comprises pressure generator 14 and a controller for pressure generator 12. Pressure generator 14 can be a pump for providing air pressure to cuff 16, thus generating pneumatic pressure in cuff 16 using the pump and valves of NIBP measurement module 26. When the supra-systolic pressure in cuff 16 is generated and maintained, pulse wave oscillations within the pressure cuff 16 are measured and transduced by pressure sensor 28. For this purpose, the Freescale MPX 2053 Case 1369-01 silicon piezoresistive pressure sensor is preferred. Other types of pressure sensors may also be used. For example, strain sensors may be attached to the outer wall of the cuff to transduce oscillations in the stretch of the cuff in response to intera-arterial pressure changes.

After the pressure sensor 28 transduces the pulse wave oscillation signals to analog electrical signals, the analog signals are processed and amplified to extract the supra-systolic oscillations of interest. As further explained below, this manipulation of the analog signal is conducted in latched amplifier 30 and the analog signal produced is then sent to A/D converter 22 where it is converted into a digital signal for further processing by digital signal processor 24.

FIG. 3 is a block diagram of a preferred embodiment of the invention. The apparatus of this embodiment is controlled by an embedded central processing unit (“CPU”) designated as Tahoe 32. Tahoe 32 interfaces with the great board 34, which in turn is connected to the other components of the apparatus. See also FIG. 4. Great board 34 contains custom signal processing electronics (as further explained below), and is connected to cuff 16 by pneumatic connector 36. Pneumatic connector 36 also connects NIBP measurement module 26 which, as previously described, controls the pneumatic pressure in cuff 16 and achieves and maintains the proper supra-systolic pressure in cuff 16. NIBP measurement module 26 can be a commercially available unit, such as supplied by Welch Allyn under the name POEM. NIBP measurement module 26 is electronically connected to great board 34, which inputs the pre-determined supra-systolic pressure information to the module 26. As shown in FIG. 3, the apparatus contains internal batteries 38 and an external DC power supply 40, and is operated by switch 42. The apparatus can optionally be connected to PC 44, interfaced through Tahoe 32.

FIG. 4 illustrates further detail of the components of great board 34. Generally, great board 34 contains components relating to power regulation and supply 48, an interface 50 to the Tahoe board 32, an interface 60 to NIBP measurement module 26, and a 100 Hz generator 52 for pacing A/D converter 22. Also, great board 34 comprises pneumatic interface 54 for pneumatic connection through pneumatic connector 36 to cuff 16. Pneumatic interface 54 is connected to pressure sensor 28 within great board 34, which measures the cuff pulse waves and provides a transduced analog signal to signal conditioner (“SCON”) 56. The output analog signal of SCON 56 is input into A/D converter 22 where it is converted into a digital signal. A/D converter 22 can be a 12 bit 16 channel A/D converter, such as AD7490.

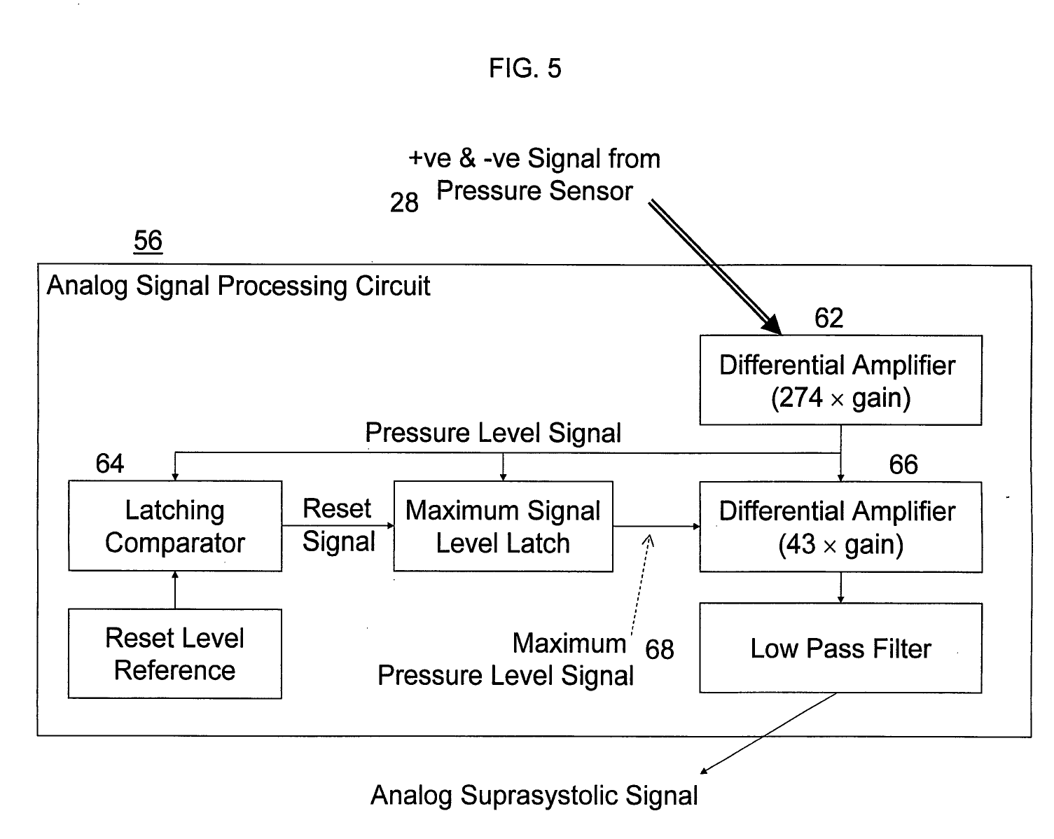

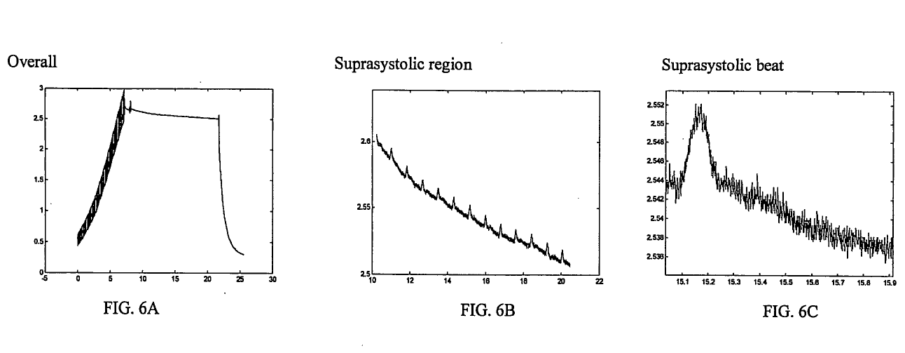

FIG. 5 is a diagram of the functions of SCON 56. Pressure sensor provides 28 a differential voltage signal proportional to the pressure it observes. This voltage ranges from approximately 0 to 5 Volts corresponding to a pressure range of 0 to 300 mmHg. However, the pressure fluctuations observed within cuff 16 (caused by the pulsatile arterial pressure) are significantly smaller than the overall voltage range. Supra-systolic pressure signals have amplitudes generally less than 0.1 V and often as small as 0.01 V. Supra-systolic pressure recordings from an oscilloscope at 16 bit resolution are shown in FIGS. 6A, 6B and 6C. If the resolution of A/D converter 22 is a typical 12-bit on a range of 5 V then each bit represents a voltage increment of 1.22 mV. In other words, far less than 100 discrete voltage levels are available to digitize the supra-systolic curve.

In order to achieve acceptable resolution from the digitization, the resolution of A/D converter 22 needs to be increased or analog amplification needs to be used to increase the voltage range seen by A/D converter 22. It is infeasible to amplify the supra-systolic analog signal relative to a 0 V reference, as the signal will quickly saturate at the upper limit of A/D converter 22. For example, in the recordings shown above, the supra-systolic analog signal is at a voltage level of approximately 2.5 V. If amplified directly by a factor of two, the supra-systolic analog signal would be twice as large (approximately 0.02 V amplitude) but sitting at a level of 5 V, which is the limit of the A/D converter 22.

It is therefore necessary to amplify the supra-systolic signal relative to a reference that is near the supra-systolic cuff pressure. In the above example, if we were to amplify the supra-systolic signal relative to a reference voltage of 2.5 V with a gain of 40, the maximum signal level would be 4.58 V (that is, (2.552−2.5)×40+2.5) and the minimum signal level 3.94 V (that is, (2.536−2.5)×40+2.5). This obviously allows much larger amplification without saturation.

Thus it is necessary to first determine the reference voltage level. The actual, “average” supra-systolic signal level is not fixed but dependent on the systolic blood pressure of the patient. The purpose of SCON circuit 56 is to determine an appropriate reference voltage and then apply amplification.

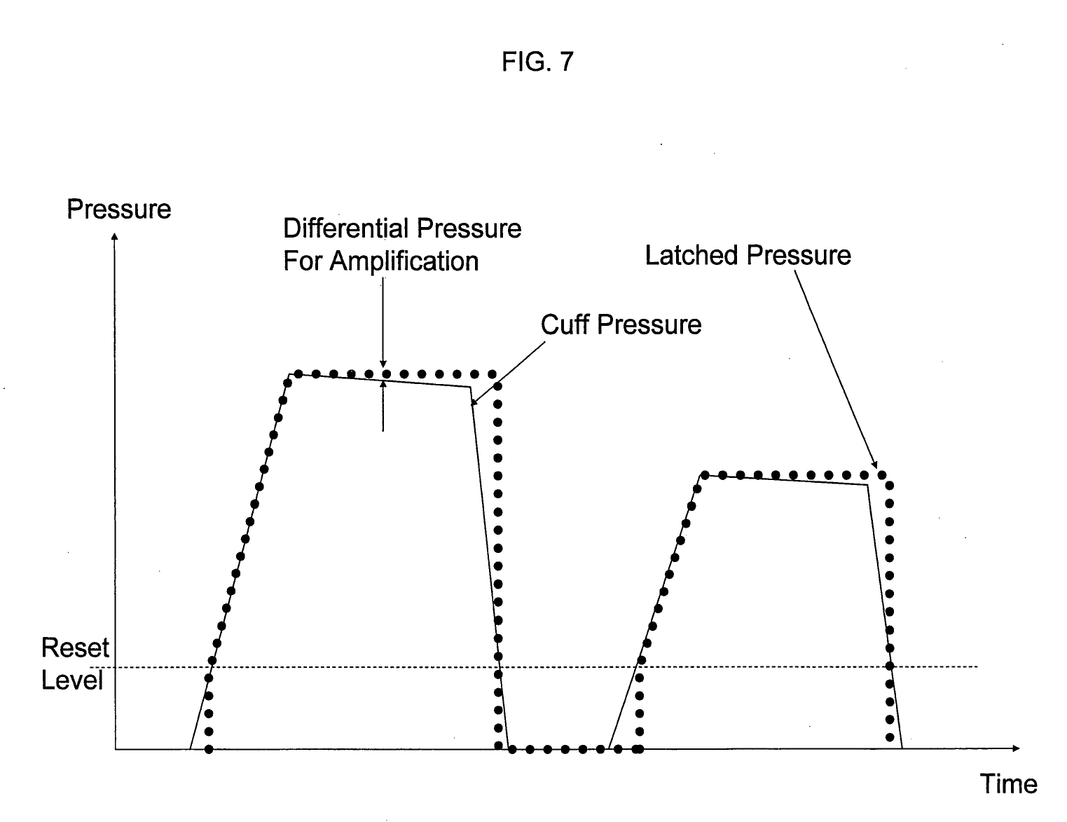

In operation, SCON circuit 56 operates in conjunction with a pressure profile for a cuff inflation cycle, an example of which is illustrated in FIG. 7. Cuff 16 is first inflated to an arbitrary pressure level to measure systolic pressure. It is then fully deflated (to a pressure below 5 mmHg) and then re-inflated to the supra-systolic pressure, calculated as approximately 30 mmHg above the systolic pressure.

The reference pressure (voltage) provided by the SCON circuit 56 (which is called the “latched voltage”) is given as the dotted line. The actual pressure in cuff 16 oscillates with each heart beat around the solid line.

While the cuff pressure remains below a predetermined reset level, the output latched voltage is zero. When the cuff pressure increases above the reset level, the output latched voltage becomes the cuff pressure voltage. As the cuff 16 deflates, the latched voltage remains at the maximum cuff pressure voltage. When the cuff pressure voltage falls below the reset level, the latched voltage then also returns to zero. In this way, the latched voltage provides a reference level corresponding to a maximum pressure. In the preferred embodiment, as the supra-systolic pressure is only ever attained by increasing the pressure in cuff 16, the latched voltage provides a suitable reference to use for differentially amplifying the supra-systolic signal.

Accordingly, the signal produced by pressure sensor 28 is input into differential amplifier 62 within SCON circuit 56, where it is amplified by a factor of 274. Referring to FIG. 5, latching comparator 64 then analyzes the amplified signal with respect to the latched voltage and the reset signal to provide a maximum pressure level signal. The maximum pressure level signal is then amplified in differential (“latching”) amplifier 66. This amplification is of interest only during a period of substantially constant cuff pressure (where that pressure is the supra-systolic measurement pressure). The gain in the latching amplifier is 43×, or equivalent to an additional 5.4-bit resolution on A/D converter 22, resulting in an equivalent 17.4-bit A/D converter resolution. The signal from differential amplifier 66 is then passed through low pass filter 68, resulting in an analog supra-systolic signal that is then digitized by A/D converter 22. As in FIG. 2, the digitized signal is further processed by digital signal processor 24.

As described, the above embodiment achieves an equivalent analog to digital conversion resolution of 17.4 bit, using a physical 12-bit converter. This effective resolution is required to extract the supra-systolic information from the pressure voltage signal, where the voltage span of about 5 V corresponds to the entire operating range of the pressure sensor (in this case, from 0 to approximately 300 mmHg).

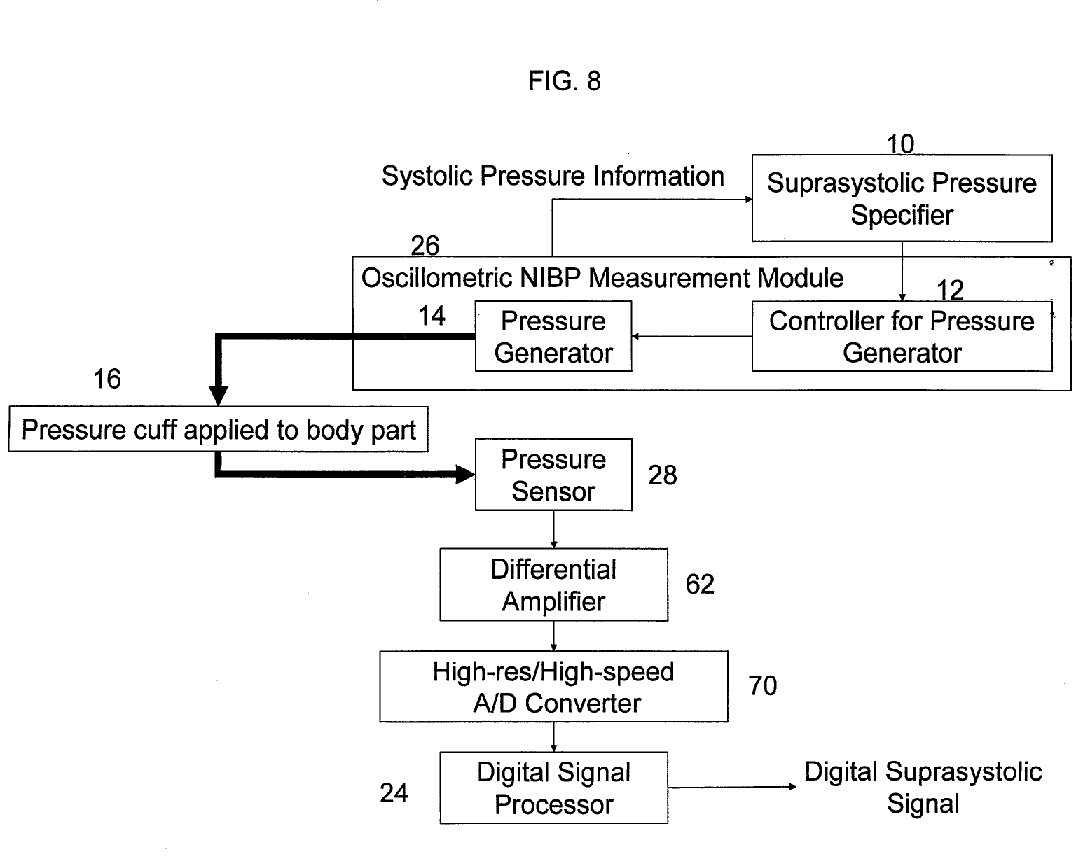

However, the same effective A/D converter resolution may be achieved using a high-resolution A/D converter, as shown in FIG. 8. This is essentially the same apparatus as shown in FIGS. 2 and 4-5, except that the transduced supra-systolic analog signal from pressure sensor 28 is input into high-resolution A/D converter 70 after being amplified 274 times by differential amplifier 62. Accordingly, there is no need for SCON circuit 56 (FIG. 4, which includes latching comparator 64, differential amplifier 66 and low pass filter 68), or latched amplifier 30 (FIG. 5). High-resolution A/D converter 70 could be an ADS1210 from Burr-Brown, which allows an effective 20-bit resolution at 1000 Hz sampling frequency. The signal from the high-resolution A/D converter 70 is then processed by digital signal processor 24 to provide a digital supra-systolic signal as previously discussed.

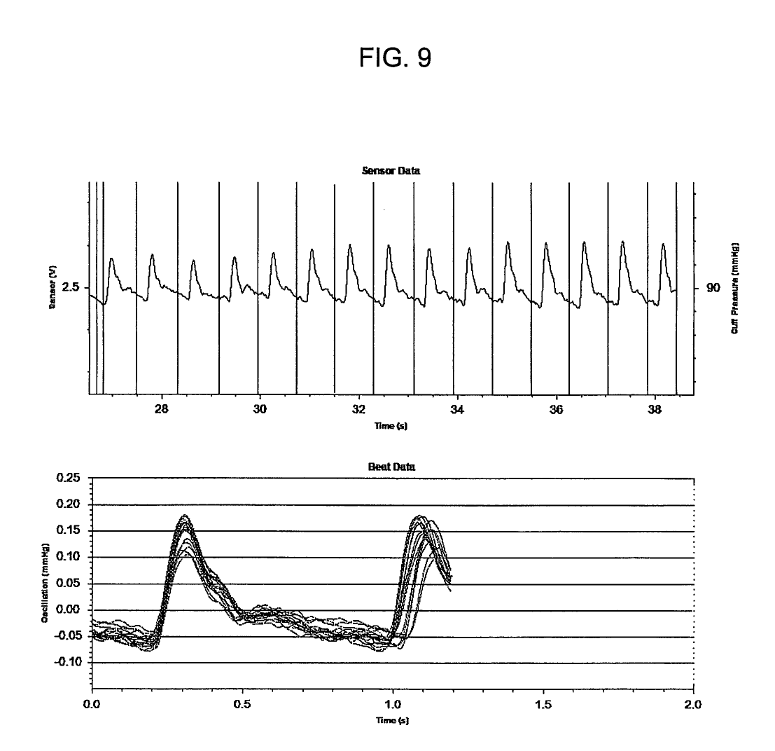

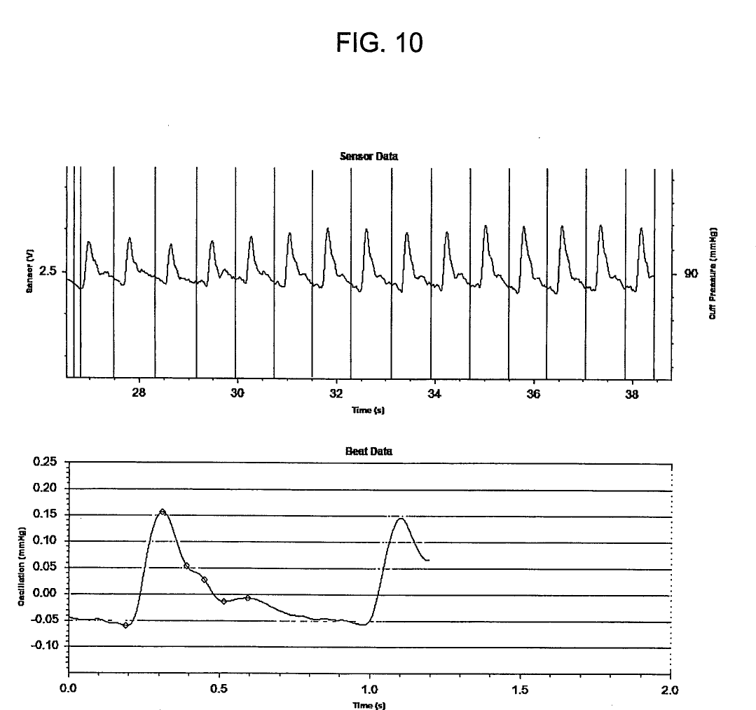

FIGS. 9 and 10 each display two charts of the digitized supra-systolic signal produced after final processing by digital signal processor 24. The upper charts in these figures show a sequence of consecutive supra-systolic beats recorded after amplification from oscillometric pressure sensor 28. Consecutive beats are shown superimposed upon one another in the lower charts. In FIG. 9, the lower chart illustrates the waveforms being overlaid, while in FIG. 10 the lower chart shows the average of the overlaid waveforms.

It can be seen that the waveforms closely resemble an intra-arterial wave and contains information about incident and reflected pressure waves, the peaks and troughs of which are shown using circled points. See FIG. 10. Transformation of this wave is able to closely reproduce the wave shape recorded using WEP sensors. See the '070 publication. Accordingly, the waveforms produced by this invention are capable of being utilized to non-invasively determine the cardiovascular status of a patient, without the need for WEP sensors.

The preceding preferred embodiments are illustrative of the practice of the invention. It is to be understood, however, that other expedients known to those of skill in the art, or disclosed herein, may be employed without departing from the spirit of the invention or the scope of the claims.

What is claimed is:

1. Method of obtaining electronic, oscillatory pressure pulse-related signals from an inflatable blood pressure cuff on a patient, said method comprising the steps of:
(a) determining systolic blood pressure of the patient;
(b) selecting a measurement pressure which is above the systolic pressure;
(c) sensing oscillatory pressure pulse-related signals at the measurement pressure to produce an electronic analog signal;
(d) converting the analog signal to a digital signal.

2. The method of claim 1, wherein the systolic pressure is determined using the blood pressure cuff.

3. The method of claim 1, further comprising the step of amplifying the analog signal prior to converting said analog signal to a digital signal.

4. The method of claim 2, wherein the brachial artery is occluded by the blood pressure cuff inflated to a supra-systolic pressure.

5. The method of claim 4, wherein the measurement pressure is a pressure in the range of substantially 25-30 mm Hg above the determined systolic pressure.

6. An apparatus for measuring an oscillometric pulse wave from a blood pressure cuff and producing a digital supra-systolic signal, comprising;
a. a blood pressure cuff applied to a human body part,
b. a pressure generator pneumatically connected to said blood pressure cuff, for generating a pre-determined supra-systolic pressure;
c. an oscillometric pulse wave sensor pneumatically connected to said blood pressure cuff for measuring oscillometric pulses wave from said blood pressure cuff and generating an analog electronic signal;
d. analog electronic circuitry for processing said analog electronic signal; and
e. analog to digital converting means for converting said analog signal to a digital signal.

7. The apparatus of claim 6, wherein said pressure generator comprises a pneumatic pump and means for controlling said pneumatic pump.

8. The apparatus of claim 7, wherein said controlling means operates said pneumatic at a pre-determined supra-systolic pressure.

9. The apparatus of claim 5, wherein said oscillometric pulse wave sensor comprises a piezoresistive pressure sensor pneumatically connected to said blood pressure cuff.

10. The apparatus of claim 5, wherein said oscillometric pulse wave sensor comprises a strain sensor mechanically connected to said blood pressure cuff.

11. The apparatus of claim 5, wherein said analog electronic circuitry comprises a differential amplifier and a latching comparator.

12. The apparatus of claim 5, wherein said analog to digital converting means produces a digital signal with at least 12 bits.

13. The apparatus of claim 5, further comprising means for measuring the systolic pressure of a patient.

14. The apparatus of claim 5, further comprising digital signal processing means.

15. An apparatus for measuring oscillometric pulse waves from a blood pressure cuff and producing a digital suprasystolic signal, comprising;
(a) a blood pressure cuff applied to a human body part,
(b) a pressure generator pneumatically connected to said blood pressure cuff, for generating a determined supra-systolic pressure;
(c) an oscillometric pressure sensor pneumatically connected to said blood pressure cuff for measuring oscillometric pulse waves from said blood pressure cuff and generating an analog electronic signal; and
(d) analog to digital converting means for converting said analog signal to a digital signal.

16. The apparatus of claim 14, wherein said analog to digital converting means comprises at least a 16 bit A/D converter.
